(19) United States
(12) Patent Application Publication
(10) Pub. No.: US 2013/0131678 A1
(43) Pub. Date: May 23, 2013

(54) THREADED ELASTIC INTRAMEDULLARY NAILS DEVICES AND METHODS

(71) Applicant: THE UNIVERSITY OF NORTH CAROLINA AT CHAPEL HILL, Chapel Hill, NC (US)

(72) Inventor: Laurence E. Dahners, Chapel Hill, NC (US)

(73) Assignee: THE UNIVERSITY OF NORTH CAROLINA AT CHAPEL HILL, Chapel Hill, NC (US)

(21) Appl. No.: 13/689,272

(22) Filed: Nov. 29, 2012

Related U.S. Application Data
(63) Continuation of application No. PCT/US2011/039100, filed on Jun. 3, 2011.
(60) Provisional application No. 61/351,106, filed on Jun. 3, 2010.

(51) Int. Cl. A61B 17/72

(52) U.S. Cl. CPC A61B 17/7208
USPC 606/62

(57) ABSTRACT

Devices and methods for treating fractures using intramedullary nails and screws are provided. An intramedullary nail and fixation device can be capable of holding a bone out to length when it is broken in a manner that allows shortening of and/or compressing a bone to promote healing. The device can include a flexible nail with a first leading end and a second end, the nail having a threaded outer surface along at least a portion of a length of the nail. A method for treatment of fractures of long bones having a medullary canal can include creating a hole into an end of a bone to be compressed, inserting a leading end of such a flexible nail into the hole, and rotating the nail to advance the nail through the medullary canal.

### CROSS REFERENCE TO RELATED APPLICATIONS

This application is a continuation of and claims priority to PCT/US20111039100 filed Jun. 3, 2011 which claims the benefit of and priority to U.S. Provisional Patent Application Ser. No. 61/351,106, filed Jun. 3, 2010, the entire disclosures of which are herein incorporated by reference.

### TECHNICAL FIELD

The subject matter disclosed herein relates generally to devices and methods for treatment of fractures of bones such as long bones having a medullary canal. More particularly, the subject matter disclosed herein relates to devices and methods for treating fractures using intramedullary nails and screws.

### BACKGROUND

Flexible intramedullary nailing of long bones is a widely-used technique in orthopedic surgery that can exhibit a number of benefits over competing treatment methods. For example, flexible nails can be inserted from the side of a bone, allowing them to be placed in children by curving them in above or below the growth plates, since it is inadvisable to insert fixation devices through the growth plates as it can arrest growth. In addition, flexible nails can be used to stabilize fractures in curved bones such as the clavicle, radius, and ulna. Another benefit can be that because intramedullary nails/rods allow the load to be shared with the bone, rather than the nails having to entirely support the bone, patients can be able to use the extremity more quickly after surgery compared to traction, plaster, or other fracture treatment options.

Despite these advantages, however, the use of intramedullary nailing can also be problematic due to issues with existing devices. Specifically, currently-used nails are often not longitudinally stable in the bone. As a result, the nails are not capable of maintaining the length of bones when fractures are configured so that the bone can shorten (e.g., comminuted, spiral fractures), where in simple fractures the bone will hold itself out to length (as long as the nail prevents translation at the fracture site). In addition, the nails can “back out” from the location in which they were inserted, which can lead to the nails becoming protuberant and causing pain. Furthermore, because of the curved path they assume in the bone, it can be difficult to “ream” the bone and enable the insertion of a larger flexible nail. As a result, the surgeon is often constrained to use a nail that is small enough to easily slide in.

Alternatively, screw fixation of fractured bone can likewise be an effective technique in orthopedic surgery. As the threads advance into the bone, they pull the head of the screw along. As a result, if the threads are on one side of a fracture and the head is on the other, they can act to pull the fracture site closed, or they can be used to pull plates down against a bone surface. Despite these advantages, fixation by way of screws or threaded pins also presents some difficulties. For instance, the screws and threaded pins on the market are commonly made to be as stiff as possible for their diameter and have sharp cutting points so that they can cut their own hole into bone. Thus, they tend to cut straight out the other side of the bone without deflecting off the inner surface of the medullary canal. In addition, the stiff screws can only follow a straight path, and thus can only travel a short distance within “flat bones” such as the pelvis, which are actually curved and shaped somewhat like a dish or bowl, before they “cut out” through a cortex. This rigidity limits the grip they can get in the bone. Further, because of this rigidity, a surgeon can generally insert a long screw within bone only if a “perfect” starting point that lines up with a long straight path in the bone is selected for the screw. In this regard, certain desirable paths within the bone are too curved and cannot be achieved at all with a rigid straight screw. Further still, the longest length in which these screws are available is about 5 inches, and threaded pins can be about 9 inches long, either of which is too short for nailing of most bones.

Accordingly, devices and methods are desired for using intramedullary nails or screws that retain the advantages of one or both of elastic intramedullary nails or bone screws while diminishing the problems of existing designs.

### SUMMARY

In accordance with this disclosure, devices and methods for treating fractures using intramedullary nails and screws are provided. In one aspect, an intramedullary nail and fixation device is provided that is configured and operable for holding a bone out to length to prevent shortening when it is broken or in a different configuration, in a manner that allows shortening of and/or compressing the bone ends together in order to promote healing. The device can comprise a flexible nail comprising a first leading end and a second end, the nail having a threaded outer surface along at least a portion of a length of the nail.

In another aspect, another intramedullary nail and fixation device is provided that can comprise a flexible nail comprising a hollow body having a first leading end and a second end, the nail having a threaded outer surface along at least a portion of a length of the nail, and a guide wire adapted to pass through the hollow body of the nail.

In yet another aspect, a method for treatment of fractures of long bones having a medullary canal is provided. The method can comprise creating a hole into an end of a bone to be compressed, inserting a leading end of a flexible nail into the hole, the nail comprising a first leading end and a second end opposite the first end, and the nail comprising a threaded outer surface along at least a portion of a length of the nail, and rotating the nail to advance the nail through the medullary canal.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described herein below.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIGS. 1A and 1B are x-ray images of threaded elastic nails being used to treat bone fractures according to an embodiment of the presently disclosed subject matter;

FIG. 2A is a side view and corresponding bottom view A-A of a threaded elastic nail according to an embodiment of the presently disclosed subject matter;

FIG. 2B is a side view and corresponding bottom view B-B of a driving tool for use with a threaded elastic nail according to an embodiment of the presently disclosed subject matter;

FIG. 3 is a side view and corresponding top view C-C of a cutting device for use with a threaded elastic nail according to an embodiment of the presently disclosed subject matter;

FIG. 4A is a side view of a threaded elastic nail according to an embodiment of the presently disclosed subject matter;

FIG. 4B is a side view of a removal tool for use with a threaded elastic nail according to an embodiment of the presently disclosed subject matter;

FIG. 5 is a side view and corresponding bottom view D-D of a driving tool for use with a threaded elastic nail according to an embodiment of the presently disclosed subject matter;

FIG. 6 is a side view of a threaded elastic nail according to an embodiment of the presently disclosed subject matter; and

FIG. 7 is a side view of an elastic screw according to an embodiment of the presently disclosed subject matter.

### DETAILED DESCRIPTION

The present subject matter provides systems and methods of treating fractures using intramedullary nails and screws. Specifically, as disclosed herein, current elastic intramedullary nails can be modified by threading their outer surface and adapting the tip of the nail to allow it to be driven with a drill or similar device rather than by impacting it with a hammer. Further and in contrast to current elastic nailing technology, threading an outer surface of these nails can allow them to grip the bone at either end, limiting bone shortening and nail “back out.” In this way, such nails can be configured and operable either for holding a bone out to length when it is broken in a manner that prevents shortening of the bone or to allow shortening of the bone to promote healing.

In addition, a similar methodology can be applied to produce “elastic screws”, which can be useful in applying “compression” across the fracture of long bones to promote healing and to repair oddly shaped bones such as the pelvis. These screws can have a blunt tip to keep them from cutting their way out of bone, a threaded section behind the blunt tip to grip the bone, a flexible shaft to allow them to follow the inner shape of the bone, and a special washer that can act as a “screwhead”. In either form, the systems and methods disclosed herein can provide advantages over existing systems and methods used in the treatment of fractures of long bones.

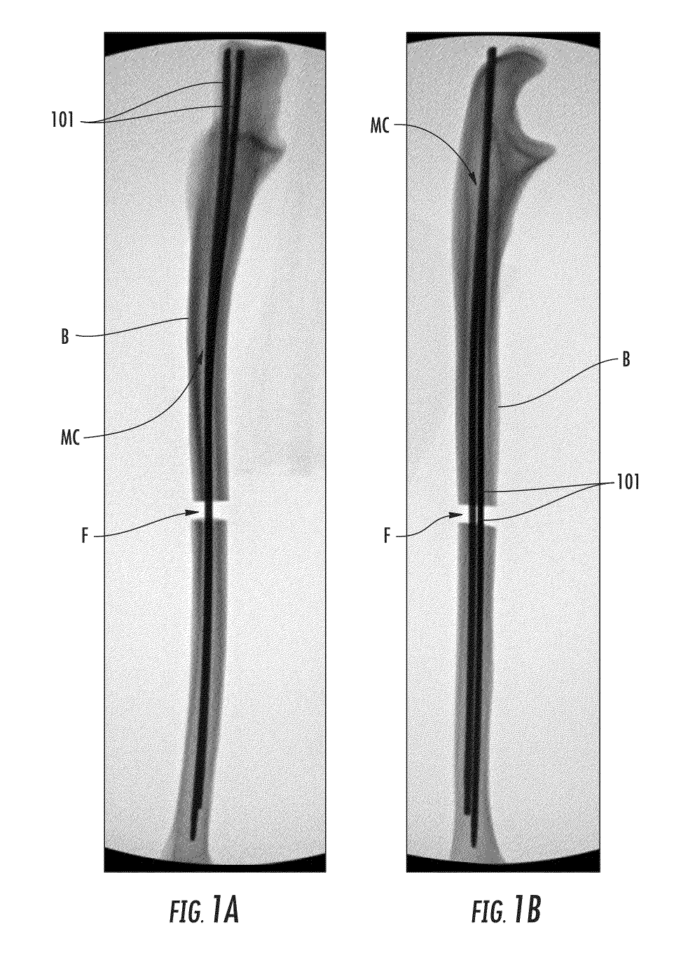

In one aspect, for example, the present subject matter provides a first threaded elastic nail that can be used in place of current elastic intramedullary nails. For example, FIGS. 1A and 1B show AP and lateral x-rays of a pair of first threaded elastic nails 101 secured in a medullary canal generally designated MC of a bone B (i.e., a cadaver ulna in FIGS. 1A and 1B), holding it to length at the defect (i.e., across a fracture site F). Similarly to current pediatric elastic nails, first threaded elastic nail 101 can be made from a material that is relatively flexible or “elastic”. For example, first threaded elastic nail 101 can be composed of titanium, certain stainless steels, especially those that have been “worked” to achieve the desired elasticity, or any of a variety of other suitable surgical implant materials. Contrary to current elastic intramedullary nail designs, however, first threaded elastic nail 101 can be threaded on the outer surface.

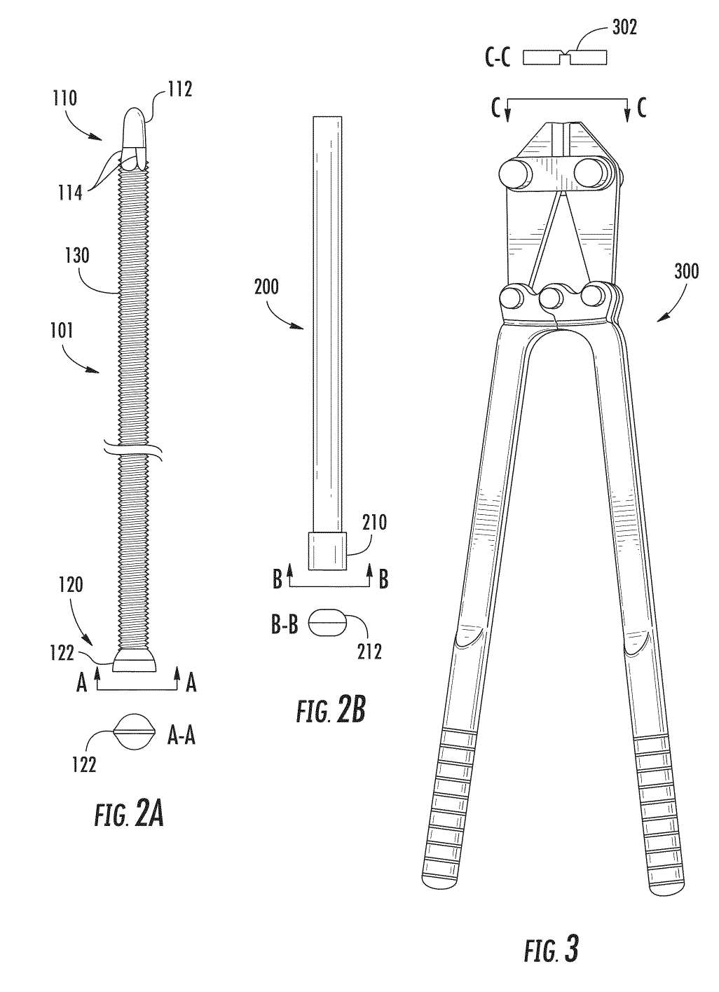

Specifically, referring to FIG. 2A for example, first threaded elastic nail 101 can comprise a first leading end 110, a second end 120 opposing first leading end generally designated 110, and a threaded outer surface 130 along substantially an entire length of first threaded elastic nail 101. A leading tip 112 of first threaded elastic nail 101 at first leading end 110 can have a substantially rounded conformation so that it will readily deflect off the inner surface of the bone rather than penetrating it. Behind rounded leading tip 112, first leading end 110 can further comprise cutting ridges 114 that can act to slightly enlarge the canal and cut threads/grooves into the inner surface of the bone being nailed. As first threaded elastic nail 101 is driven into the canal, these grooves can be engaged by threaded outer surface 130 to thereby facilitate the drilling action of first threaded elastic nail 101. In other words, this threading into the opening of the bone can cause first elastic nail 101 to advance steadily into the bone as it is rotated. Threaded outer surface 130 can further prevent first threaded elastic nail 101 from backing out and becoming painful.

First threaded elastic nail 101 can have a diameter that is slender enough so that first threaded elastic nail 101 exhibits flexibility. For example and without limitation, first threaded elastic nail 101 can have a diameter of between approximately 2.4 mm and 3.6 mm. With these proportions, first threaded elastic nail 101 can remain substantially elastic. Of course, a person having ordinary skill in the art will recognize that a slender body having a larger diameter can still retain some measure of flexibility, but nails having much larger diameters can also become too stiff for the intended purpose described herein and can cause problems with heating and breakage as surgeons try to pass them in such a fashion that they have too much curve in them for a thick nail to tolerate. Accordingly, the desire for a thicker body can be weighed against these factors in determining a desired thickness for first threaded elastic nail 101.

Initial insertion and advancement of first threaded elastic nail 101 into the bone can be accomplished by chucking first threaded elastic nail 101 directly into a power driving tool (e.g., an electric drill) that can rotate it so that it is advanced into the bone. Because it is desirable to have the nail protrude only a short distance out of the bone so that it is not prominent and painful, however, when first threaded elastic nail 101 is nearly fully inserted, it can be driven the remaining distance (e.g., a few centimeters) with a specialty driving tool. Referring to FIG. 2B, for example, a driving tool 200 that is designed to engage first threaded elastic nail 101 can be used to finish driving first threaded elastic nail 101 into the target bone. To engage driving tool 200 with first threaded elastic nail 101, the contacting ends of each component can be designed to have any suitable complementary shape. For instance, referring again to FIG. 2A, second end 120 of first threaded elastic nail 101 can be shaped to define a first profile 122. Specifically, for example, end view A-A of second end 120 shows that first profile 122 can comprise a shape that tapers to a substantially straight edge similar to the shape of a screwdriver blade. To complement this shape, driving tool 200 can comprise a first end 210 having a second profile 212. As shown in view B-B of first end 210, second profile 212 can comprise a shape that defines a slot (e.g., a substantially V-shaped indentation) that can operate as a socket to engage the flat, blade-shaped first profile 122 of second end 120 of first threaded elastic nail 101.

With this configuration, first profile 122 can be coupled to second profile 212 such that rotation of driving tool 200 can cause rotation of first threaded elastic nail 101, thereby allowing the surgeon to drive first threaded elastic nail 101 into the target bone through a small incision (and later remove it through a small incision). Driving tool 200 can be rotated by hand, or it can be coupled to a powered tool, such as an electric drill. It should be understood to those have skill in the art, that shapes for first profile 122 other than a straight blade (e.g., a triangular cross-section) can be used to achieve similar results when used with a driving tool having a complementarily-shaped profile.

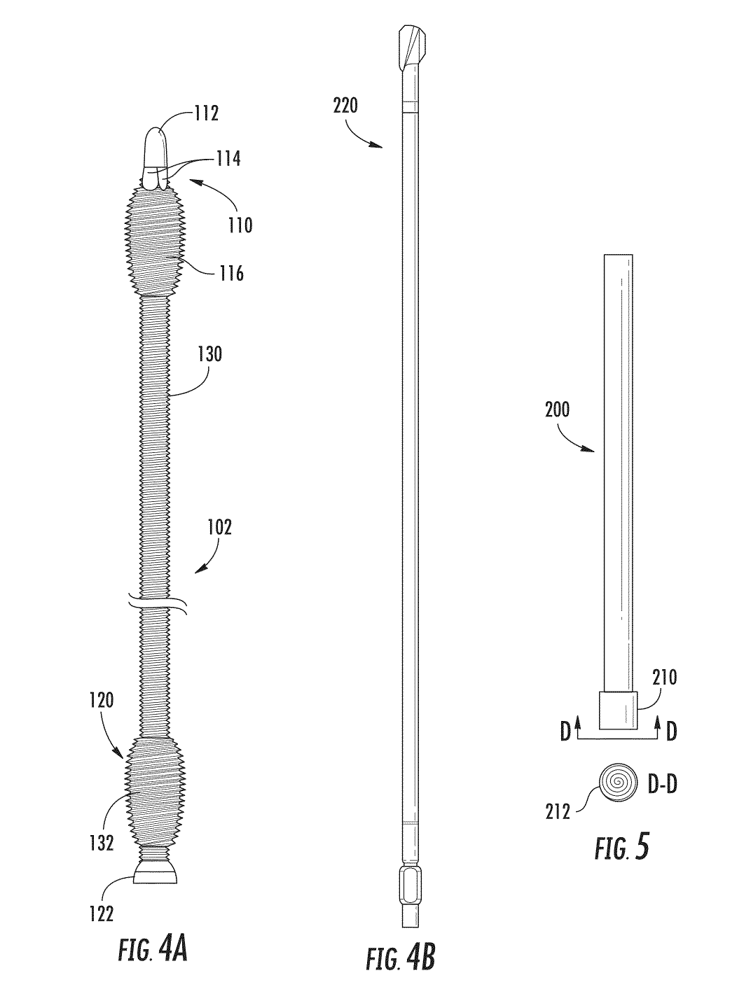

Referring to FIG. 3, a cutting device 300 can be used to cut first threaded elastic nail 101 to have a desired length. In this way, first threaded elastic nail 101 can be shortened based on the specific circumstances of each individual use (e.g., patient bone length, position of fracture). In addition, cutting device 300 can define a cutter head 302 that can have a desired profile, such as for example and without limitation the profile shown in section view C-C. In particular, cutter head 302 can be shaped to deform second end 120 of first threaded elastic nail 101 as it cuts so that second end 120 has the desired shape of first profile 122. As a result, even if first threaded elastic nail 101 needs to be cut to a particular length, no further modification needs to be made to allow first end 210 of driving tool 200 to cooperate with second end 120 of first threaded elastic nail 101 to engage the two components for rotation together. With this configuration, once any excess length is cut off, first threaded elastic nail 101 can be driven until it is nearly flush with the bone surface through a small incision (i.e., rather than driving it to its full depth and then making a large incision for introduction of a cutter in order to cut it to be nearly flush).

Using first threaded elastic nail 101 having the configuration discussed above, a method for a long bone having a medullary canal can comprise making a small incision near an end of the bone. Most bones have a wide end (e.g., medial clavicle, proximal ulna, distal radius, distal femur, proximal tibia, distal fibula) and a narrow end, so the small incision can be made at the wide end, for example. A trochar and drill sleeve can then be introduced down to the bone and a spike at the tip of the sleeve can be driven into the bone to hold it there (after which point, the trochar can be removed). A drill slightly smaller than the outer thread diameter of first threaded elastic nail 101 can be used to make a starter hole, which can be slanted very obliquely into the medullary canal of the bone. Rounded leading tip 112 of first threaded elastic nail 101 can be passed down the drill sleeve and “nosed” into the hole, and a power drill can be used to rotate first threaded elastic nail 101 so that it threads itself into the hole. In some embodiments, a “paired” drill sleeve (i.e., a sleeve having multiple guide slots) can be used to assist in drilling for and placement of additional threaded nails adjacent to first threaded elastic nail 101. Such a compound sleeve can aid in the placement of multiple nails in a controlled grouping.

As discussed above, rounded leading tip 112 can allow first threaded elastic nail 101 to deflect off the inner surface of the bone. In combination with the elastic nature of the material used to form the body of first threaded elastic nail 101, rounded leading tip 112 can thus allow first threaded elastic nail 101 to travel and flex as needed in a curving path down the inside of the medullary canal of the bone despite the constant rotation of drilling it in. In contrast, the metallurgy of current threaded pins is so stiff that they would bend (i.e., plastically deform into a new retained shape rather than elastically deform to a shape from which they can spring back) rather than flex as they travel or advance down the inside of a curved bone. Such pins would then markedly resist rotation by the drill that was attempting to thread them further into the bone, which would likely lead to eventual breakage of such pins. Stiff pins can also heat as they are rotated, bent, and/or deformed by the driver, which is undesirable.

When the tip of first threaded elastic nail 101 arrives at the fracture site, the surgeon can then reduce the fracture while the drill can advance first threaded elastic nail 101 across the fracture into the other end of the bone. Advancing first threaded elastic nail 101 across the fracture site in this manner (i.e., by rotating it with a drill) can be done in a much more controlled manner compared to driving it across with a hammer as is the current practice with flexible nails. First threaded elastic nail 101 can then be advanced until either threaded outer surface 130 obtains a good bite in the narrow end of the bone, or until it is well within the narrow end. If a good bite is not obtained, then one or more additional first threaded elastic nails 101 can be also be inserted into the bone until a tight fit in the narrow end of the bone is obtained.

With one or more first threaded elastic nails 101 being threaded into the bone on both ends, the bone can be held out to the length it has at the time of installation of the nails, which can help the bone to resist shortening and resist backing out of first threaded elastic nails 101, which can promote healing at the correct length. In addition, the use of more than one of first threaded elastic nails 101 (i.e., in larger bones) can also provide resistance to rotational deformations. Further, in certain embodiments where multiple nails are used, each of the multiple first threaded elastic nails 101 can have the same thread pitch so that when they are screwed into the medullary canal next to one another, their threads can intermesh with each other to essentially “lock” one against the other. In this way, several of first threaded elastic nails 101 can act much like one larger fixation device.

By comparison, placement of current flexible nails large enough to fit tightly into the bone is nearly impossible because they will not readily advance down a curving canal and instead become “jammed.” This jamming can be overcome by opening the fracture site through a separate, often relatively large incision to drill the inside of the bone up to a larger diameter, thus allowing passage of the nail. Such additional incisions and dissections are highly undesirable, however, and this procedure generally results in the use of nails that are smaller and weaker than desired. In contrast, once rounded leading tip 112 of first threaded elastic nail 101 finds its way into the bone, cutting ridges 114 can enlarge the hole (e.g., somewhat like a drill or reamer), thus obtaining a tight fit with a larger nail. This feature allows first threaded elastic nail 101 to be comparatively larger than smooth nails that must be advanced with hammering.

It is recognized, however, that first threaded elastic nail 101 may not be able to maintain length in larger bones such as the femur and tibia because the narrow diameter of first threaded elastic nail 101 that allows the device to be flexible enough to insert into curved shapes may be insufficient to impose sufficient forces on large-diameter bones, and inserting multiple of first threaded elastic nails 101 to fill the canal in a larger bone can require drilling more holes than would be recommended.

Accordingly, in another aspect, the present subject matter provides a second threaded elastic nail, generally designated 102, that can be used in a manner similar to the threaded elastic nail described above. Second threaded elastic nail 102 can be configured to have many features that are substantially similar to first threaded elastic nail 101, and thus the same reference numbers are used to identify these similar features. In particular, similarly to first threaded elastic nail 101, second threaded elastic nail 102 can comprise a first leading end 110 having a rounded leading tip 112 and cutting ridges 114, a second end 120 having a first profile 122, and a threaded outer surface 130.

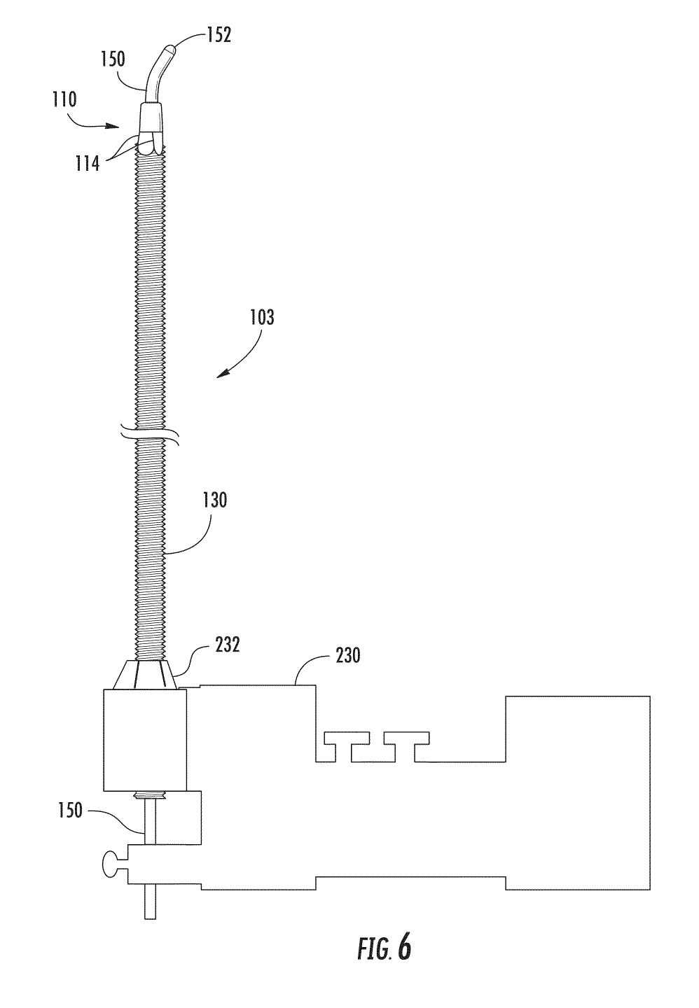

In addition, as shown in FIG. 4A, second threaded elastic nail 102 can further comprise an enlarged end portion 116 at first leading end 110, which can have a larger threaded area to help fill the canal (e.g., having a diameter that is greater than the diameter of the body of second threaded elastic nail 102), allowing first leading end 110 to grip into larger bones. Similarly, second threaded elastic nail 102 can also comprise a threaded overscrew 132 that can be attached to second end 120 to likewise grip an enlarged opening at the entrance cortex of the target bone and prevent shortening. With this configuration, enlarged end portion 116 can fill the distal canal, and threaded overscrew 132 can fill the enlarged entry hole, while the comparatively thinner flexible body (i.e., the central portion of second threaded elastic nail 102 having threaded outer surface 130) can still be flexible enough to follow the curves of entry to the canal and the curved shape of the shaft of the bone.

It is noted, however, that after healing, the large tip of second threaded elastic nail 102 may be difficult to extract, so it is desirable that their shaft be of a dimension over which a flexible reamer 220 (See, e.g., FIG. 4B) could be passed to clear the “back path” for enlarged end portion 116. Alternatively, second threaded elastic nail 102 can be designed to cooperate with tools that are commonly used in current practice. Specifically, for example, second threaded elastic nail 102 can be designed to have a diameter of approximately 3.2 mm so that a standard reamer can be used to remove second threaded elastic nail 102 by someone not having access to flexible reamer 220. In yet a further alternative, however, multiple sizes and configurations for second threaded elastic nail 102 can be used in combination without the need for specialty tools. In one particular example, one or more of second threaded elastic nail 102 having an enlarged size (e.g., enlarged end portion 116 can have a diameter of approximately 5 mm such that two of them would fill a 10 mm canal), and additional second threaded elastic nails 102 or first threaded elastic nails 101 having more conventional sizes (e.g., having diameters of approximately 3.2 mm or 3.6 mm) can be used to bind in tightly with the larger versions.

In addition, an alternative form for driving tool 200 can be used in place of or in combination with the embodiment of driving tool 200 shown in FIG. 2B in situations where first profile 122 of first threaded elastic nail 101 or second threaded elastic nail 102 is damaged or otherwise fails, either during driving or removal. As shown in FIG. 5, driving tool 200 can be designed to have a shape that can interact with second end 120 of first threaded elastic nail 101 or second threaded elastic nail 102 without using a correspondingly-shaped coupling profile. Specifically, for example, driving tool 200 can be a hardened tool in which second profile 212 defines a conical threaded hole at first end 210. Such a conical threaded hole can cut into second end 120 of first threaded elastic nail 101 or second threaded elastic nail 102 and bind to the material of second end 120 as the cone narrows. This binding can allow the surgeon to advance or back out a nail with a poorly formed second end 120. Further in this regard, the conical threaded hole of second profile 212 in this configuration can be right-hand threaded where driving tool 200 is used to advance the nails, or it can be left-hand threaded where driving tool 200 is used to back the nails out. For nail removal, second end 210 can further be adapted to have cutting “saw” teeth that, during removal of first threaded elastic nail 101, can further enable second end 210 to cut through bone down to the point that it enables profile 212 to bind to the material of second end 120.

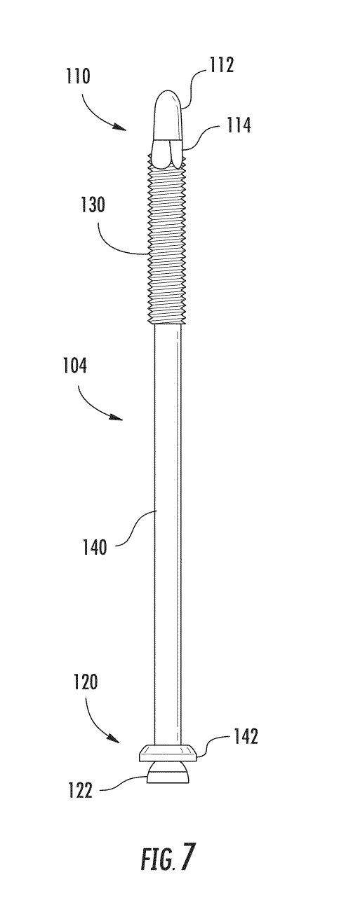

In yet another aspect, the present subject matter provides a third threaded elastic nail, generally designated 103, that can incorporate many of the features described herein above into a cannulated nail configuration. Specifically, referring to FIG. 6, third threaded elastic nail 103 can comprise a first leading end 110 having cutting ridges 114 and a threaded outer surface 130. Rather than having a rounded leading tip, third threaded elastic nail 103 can comprise a hollow body throughout its length to allow a guide wire 150 to pass through third threaded elastic nail 103. Guide wire 150 can extend from first leading end 110, with guide wire 150 comprising a curved tip 152. Further, curved tip 152 can be shaped to bend in one direction, and a back end of guide wire 150 can be rotated within the hollow bore of third threaded elastic nail 103 to control the direction of bend of curved tip 152, thereby allowing guide wire 150 to be “steered” to follow a preferred path through the bone. For example, the back end of guide wire 150 can comprise an angled portion or other form of grippable feature to allow it to be grasped and rotated to control the direction of curved tip 152.

With this configuration, a cannulated nail driver 230 can be used screw third threaded elastic nail 103 into place. Cannulated nail driver 230 can be attached to both third threaded elastic nail 103 (e.g., by clamping onto third threaded elastic nail 103 using an offset chuck 232) and guide wire 150. This connection to both elements can allow cannulated nail driver 230 to push guide wire 150 ahead of third threaded elastic nail 103 as third threaded elastic nail 103 is driven in. The surgeon can rotate cannulated nail driver 230 to change the direction of curved tip 152 (because the guide wire 150 is gripped firmly by cannulated nail driver 230), allowing the surgeon to “steer” the nail around curves and past obstacles within the bone as it threads itself into place.

Alternatively, another method for achieving a similar result can involve driving a threaded flexible shaft with a curved tip through an enlarged entry hole into the bone along a chosen path (i.e., driving it with a mallet similarly to current unthreaded flexible nails). The surgeon can use the curved tip of the shaft to direct it along a path of the surgeon's choosing in a manner similar to the steering of guide wire 150. Once the shaft is in place, an “overscrew” can be threaded in over the shaft (e.g., a cannulated nail such as third threaded elastic nail 103), driving it into place with a driving tool adapted to fit over the flexible shaft. A bead at the tip of the flexible shaft (not shown) can prevent the overscrew from running off the end of the shaft. Next, a washer or another overscrew (e.g., a threaded overscrew 132 like that disclosed for use with second threaded elastic nail 102) can be passed onto the trailing end of the flexible shaft to hold the bone at a desired length, or instead a nut can be threaded onto the shaft to compress the fracture.

In either form, insertion across the fracture site can be eased compared to solid-body nails that are driven without the assistance of a guide wire or flexible shaft. It is recognized, however, that the manufacture of third threaded elastic nail 103 can involve different considerations than the production of other embodiments discussed above. For example, thick-walled titanium tubing can be rolled to a desired size, and the rolled tubing can be cut to form threaded outer surface 130.

In still another aspect, the present subject matter provides an elastic bone screw, generally designated 104, which can likewise incorporate many of the features described hereinabove. Referring to FIG. 7, elastic bone screw 104 can comprise a first leading end 110 having a rounded leading tip 112 and cutting ridges 114, a second end 120, and a threaded outer surface 130. Rather than threaded outer surface 130 being formed along substantially its entire length in a manner similar to the configurations of the threaded elastic nails discussed herein, however, threaded outer surface 130 of elastic bone screw 104 can extend over only a portion of its length (e.g., a portion near first leading end 110 as shown in FIG. 7 without extending half the distance between the opposing end portions), with elastic bone screw 104 having an unthreaded shank portion and an unthreaded outer surface 140 over the remainder of its length that can be comparatively smooth. In this configuration, whereas threaded outer surface 130 can resist shortening and backing out of the device, unthreaded outer surface 140 can allow for compression at the fracture site of the bone.

Elastic bone screw 104 can be configured to have a conventional screw head at second end 120, and therefore can be produced in a number of different lengths. In this configuration, however, the surgeon may be required to either guess the length of the screw that is needed or drill and measure the depth of a hole for the screw before implantation. The latter option can be problematic, as drills are typically rigid they will not follow a curved path, and requiring a procedure involving introducing first a drill, then a depth gauge, and then a screw along the curved path can be very complex.

Thus, as an alternative, second end 120 can be cut to length in a manner similar to the cutting of first threaded elastic nail 101 discussed above. Specifically, a cutting device 300 can be used to cut elastic bone screw 104 to have a desired length, and cutting device 300 can define a cutter head 302 shaped to deform second end 120 of elastic bone screw 104 as it cuts so that second end 120 has a first profile 122. Because threaded outer surface 130 of elastic bone screw 104 does not extend all the way to second end 120, a washer 142 can be positioned at second end 120 to act as a screw head to help prevent elastic bone screw 104 from being driven too deep into the bone (i.e., driven to a point at which second end 120 is below an outer surface of the bone) and/or to exert a force to thereby apply compression at the fracture site of bones fractured in such a simple manner that they will not shorten excessively (i.e., such that compression can promote healing). With this configuration, elastic bone screw 104 can be cut to length shortly before it reaches its full insertion depth. When cut, second end 120 of elastic bone screw 104 can be deformed/enlarged such that washer 142 cannot slip back over second end 120, thus allowing washer 142 to act as a screwhead as discussed above.

Advantageously, elastic bone screw 104 can be designed to bend and follow a curved path through a target bone. Thus, in one particular example, elastic bone screw ‘104 can be implanted between the tables of a bone such as the pelvis in order to act in some ways like a nail and in some ways like a screw. Using conventional devices, a surgeon would need to install a lag screw to apply compression, and the surgeon must then drill a larger hole in the near bone fragment unless the bone is large enough to hold a 6.5 mm screw (many areas of the pelvis are not large enough) because the standard smaller 4.0 mm lag screws are not long enough for pelvic fractures.

In contrast, using elastic bone screw 104 as described hereinabove, a surgeon can percutaneously repair a pubic ramus fracture, for example by making an incision somewhat coaxially to the medullary canal of the ramus. Then, a trochar and drill sleeve can be introduced down to the bone at a point as close as possible to an alignment coaxial to the medullary canal and a spike at the tip of the sleeve driven into bone to hold it there, at which point the trochar could be removed. A drill can be used to perforate the cortex and elastic bone screw 104 can be introduced into the hole through the sleeve. A power driver can be used to rotate elastic bone screw 104, advancing it until rounded leading tip 112 reaches the fracture site. The fracture can be reduced, either in a closed fashion by manipulation or by opening the fracture (but with a much smaller incision than is required to plate the fracture).

Elastic bone screw 104 can then be advanced across the fracture and the surgeon can either estimate how much farther it could be driven into the bone or drive elastic bone screw 104 to the desired depth and then back it out. If desired, one or more additional elastic bone screws 104 can be placed alongside the first using parallel drill guide sleeves. Second end 120 of elastic bone screw 104 can have washer 142 advanced over it and be cut off at the appropriate level with a tool (e.g., cutting device 300) that would crimp second end 120 to have first profile 122. In this way, elastic bone screws 104 can be cut to an appropriate length after they are mostly implanted, saving the surgeon many steps. In addition, this customizable length of elastic bone screws 104 can help to markedly reduce inventory compared to the hundreds of screws of different lengths necessary for current surgical procedures. Once elastic bone screw 104 is cut to the desired length and first profile 122 is formed, driving tool 200 can then be placed over first profile 122 and used to advance elastic bone screw 104 in to the desired depth using washer 142 to compress the fracture.

In this way, elastic bone screw 104 can provide several advantages over current bone screws. First, for example, rounded leading tip 112 of elastic bone screw 104 can deflect off the inside of the bone. For example, rounded leading tip 112 can deflect off of the outer cortex, or in the case of use on the ramus, it can also deflect off the subchondral bone of the acetabulum preventing inadvertent penetration of the hip joint. Further, the elastic nature of elastic bone screw 104 can allow it to travel a curving path down the inside of the bone despite the constant rotation used to drive it into place. By comparison, the metallurgy of current screws is stiff so that they bend (i.e., plastically deform into a new retained shape rather than elastically deform to a shape from which they can spring back) rather than flex. Thus, as current screws curve with the bone and become bent they then would markedly resist rotation by the drill and would likely break. Stiff screws can also heat as they are rotated, bent, and/or deformed by the driver, which can be undesirable to the surrounding tissue even if it does not weaken the screw.

The present subject matter can be embodied in other forms without departure from the spirit and essential characteristics thereof. The embodiments described therefore are to be considered in all respects as illustrative and not restrictive. Accordingly, features of one embodiment described above can be applied equally to further embodiments. As a result, although the present subject matter has been described in terms of certain preferred embodiments, other embodiments that are apparent to those of ordinary skill in the art are also within the scope of the present subject matter.

What is claimed is:

1. An intramedullary nail fixation device for insertion into a bone having a medullary canal, the device comprising a flexible nail comprising a first leading end and a second end, the nail having a threaded outer surface along at least a portion of a length of the nail, wherein the nail is configured and operable for holding a bone out to length when it is broken in a manner that allows shortening of and/or compressing the bone to promote healing.

2. The intramedullary nail fixation device of claim 1, wherein the nail comprises titanium, stainless steel, or another suitable surgical implant material.

3. The intramedullary nail fixation device of claim 1, wherein the first leading end of the nail comprises a substantially rounded tip.

4. The intramedullary nail fixation device of claim 3, wherein the first leading end of the nail comprises one or more ridges behind the substantially rounded tip, the one or more ridges being adapted for cutting grooves to be engaged by the threaded outer surface of the nail.

5. The intramedullary nail fixation device of claim 1, wherein the second end of the nail has a shape corresponding to a shape of a socket of a driving tool.

6. The intramedullary nail fixation device of claim 5, wherein the second end of the nail has a shape that tapers to a substantially straight edge corresponding to a slot of a driving tool.

7. The intramedullary nail fixation device of claim 5, wherein the second end of the nail has a triangular cross-sectional shape corresponding to a triangular socket of a driving tool.

8. The intramedullary nail fixation device of claim 5, wherein the driving tool is comprises a drill chuck.

9. The intramedullary nail fixation device of claim 1, wherein the threaded outer surface extends along at least substantially an entire length of the nail.

10. The intramedullary nail fixation device of claim 1, wherein the threaded outer surface extends along a portion of the length of the nail near the first leading end and wherein the nail comprises a smooth, unthreaded outer surface along a portion of the length of the nail behind the portion having the threaded outer surface.

11. The intramedullary nail fixation device of claim 1, comprising an enlarged end portion at the first leading end, the enlarged end portion having a diameter that is greater than a diameter of the nail.

12. The intramedullary nail fixation device of claim 1, comprising a threaded overscrew adapted to be attached to the second end of the nail, the threaded overscrew having a diameter that is greater than a diameter of the nail.

13. An intramedullary nail fixation device capable of holding a bone out to length when it is broken in a manner that prevents shortening of the bone or compressing the bone to promote healing, the device comprising:
a flexible nail comprising a hollow body having a first leading end and a second end, the nail having a threaded outer surface along at least a portion of a length of the nail; and
a guide wire adapted to pass through the hollow body of the nail.

14. The intramedullary nail fixation device of claim 13, wherein the guide wire comprises a substantially curved tip.

15. A method for treating fractures of long bones having a medullary canal, the method comprising:
creating a hole into an end of a bone to be compressed;
inserting a leading end of a flexible nail into the hole, the nail comprising a first leading end and a second end opposite the first end, and the nail comprising a threaded outer surface along at least a portion of a length of the nail; and
rotating the nail to advance the nail through the medullary canal.

16. The method of claim 15, wherein the first leading end of the nail comprises a substantially rounded tip, wherein the first leading end deflects away from an inner surface of the bone such that the nail is maintained in the medullary canal as it is advanced.

17. The method of claim 16, wherein the first leading end of the nail comprises one or more ridges behind the substantially rounded tip, and wherein rotating the nail causes the one or more ridges to create grooves in the inner surface of the bone.

18. The method of claim 15, wherein rotating the nail comprises engaging the second end of the nail with a drill and operating the drill to advance the nail.

19. The method of claim 15, wherein rotating the nail comprises engaging the second end of the nail with a socket of a driving tool, wherein the second end of the nail has a shape corresponding to a shape of the socket.

20. The method of claim 15, wherein rotating the nail comprises engaging the second end of the nail with a socket of a driving tool, wherein the socket has a conical threaded hole adapted to cut into the second end of the nail and bind to the second end.

21. The method of claim 15, comprising inserting additional flexible nails into the bone and rotating the additional nails to advance the additional nails through the medullary canal.

22. A method for treating fractures of long bones comprising:
inserting a leading end of a flexible nail into a bone, the nail comprising a first leading end and a second end opposite the first end, the first leading end comprising a threaded outer surface, and the nail comprising a head attached to the second end; and
screwing the first leading end of the nail into the bone;
wherein the head attached to the second end applies compression at a fracture site of the bone.
